(19) United States
(12) Patent Application Publication
(10) Pub. No.: US 2026/0357171 A1
(43) Pub. Date: Nov. 12, 2026

(54) MICRODEVICES FOR USE IN CELL SEEDING ONTO TWO- AND THREE-DIMENSIONAL BIOCOMPATIBLE CONSTRUCTS

(71) Applicant: Rutgers, The State University of New Jersey, New Brunswick, NJ (US)

(72) Inventors: Jeremy M. Perrelle, New Brunswick, NJ (US); Amir Mccormick, East Brunswick, NJ (US); Jasmine M. Gamboa, Union City, NJ (US); Andrew J. Boreland, Annandale, NJ (US); N. Sanjeeva Murthy, Princeton, NJ (US); Zhiping Pang, New Providence, NJ (US)

(73) Assignee: Rutgers, The State University of New Jersey, New Brunswick, NJ (US)

(21) Appl. No.: 19/116,700

(22) PCT Filed: Sep. 27, 2023
(86) PCT No.: PCT/US2023/075223

§ 371 Date: Mar. 28, 2025

Related U.S. Application Data
(60) Provisional application No. 63/377,467, filed on Sep. 28, 2022.

(51) Int. Cl. C12M 1/12; C12M 3/00

(52) U.S. Cl. CPC C12M 23/06; C12M 23/46; C12M 25/14

(57) ABSTRACT

This disclosure provides microdevices and methods of using the microdevices to grow and seed cells onto various biocompatible materials such that those cells can later be successfully engrafted into an animal (e.g., human).

### CROSS-REFERENCE TO RELATED APPLICATIONS

This application claims the benefit under 35 U.S.C. § 119(e) of the earlier filing date of U.S. Provisional Patent No. 63/377,467, filed on Sep. 28, 2022, which is hereby incorporated by reference in its entirety.

### FIELD OF THE INVENTION

The present invention relates to microdevices and methods of using the microdevices to grow and seed cells onto various biocompatible materials.

### BACKGROUND

Recent developments in cell culturing technology have made the development of in vitro cell lines and tissues a prominent area of research within the field of regenerative medicine. While autologous and allogeneic transplants have found utility from host-host skin grafts to donor-host bone grafts, and myriad other procedures, development of in vitro tissues is gaining traction, as man-made tissues can have greater tunability and customization properties than these in vivo sources of transplant materials.

Despite such advances, the field of micro-bioengineering, to promote the use of such cells and tissues, is still nascent in its ability to recapitulate the full complexity of human tissue and enable in vitro products to be engrafted effectively.

### SUMMARY OF THE INVENTION

In one aspect, this disclosure provides microdevices and methods of using the microdevices to grow and seed cells onto various biocompatible materials such that those cells can later be successfully engrafted into an animal (e.g., human). In another aspect, this disclosure provides a snap-fit fixture comprising a large snap-fit fixture and a small snap-fit fixture, wherein the large snap-fit fixture and the small snap-fit fixture detachably and reversibly connect to create a platform.

In some embodiments, the platform is configured to hold a biocompatible polymer material that can be seeded on one or both sides with cells.

In some embodiments, the large snap-fit fixture and the small snap-fit fixture are made from polylactic acid (PLA), polycaprolactone (PCL), polyvinyl alcohol (PVA), or any combination thereof.

In some embodiments, the large snap-fit fixture is about 2 mm to about 20 mm in height, and about 5 mm to about 40 mm in diameter. In some embodiments, the large snap-fit fixture comprises an aperture in the center of the large snap-fit fixture, optionally about 3 mm to about 30 mm in diameter.

In some embodiments, the small snap-fit fixture is about 2 mm to about 20 mm in height, and about 5 mm to about 40 mm in diameter. In some embodiments, the small snap-fit fixture comprises an aperture in the center of the small snap-fit fixture, optionally about 3 mm to about 30 mm in diameter.

In some embodiments, the snap-fit fixture has a weight of about 0.25 grams to about 5 grams.

In some embodiments, the small snap-fit fixture comprises outwardly protruding tabs, optionally placed on opposite sides of the small snap-fit fixture.

In some embodiments, the large snap-fit fixture comprises outwardly protruding tabs, optionally placed on opposite sides of the large snap-fit fixture.

In some embodiments, the wherein the outwardly protruding tabs comprise insets.

In some embodiments, the large snap-fit fixture comprises holes and/or openings that extend from the outside of the fixture into the interior space.

In one aspect, this disclosure provides a method of using the snap-fit fixture of any of the preceding claims to facilitate the adhesion and/or growth of cells onto one or both of the snap-fit fixtures and/or biocompatible polymers locked in the snap-fit fixture.

In some embodiments, provided is a method of using the snap-fit fixture as described herein to lock in place a biocompatible polymer material and seed that polymer material on one or both sides with cells of similar or different tissue identities.

In some embodiments, provided is a method of using the snap-fit fixture as described herein to create biomaterial scaffolds of multiple layers of polymer sheets and cells.

In one aspect, this disclosure provides a 3D column microdevice comprising: a barrel, a center column, a gear-shaped ring with a central depression inset, and a gear-shaped ring with a patent opening.

In some embodiments, the center column is capable of being inserted through the central hole in the gear; the ring is configured to slide down to the flange on the column; and a tubular biomaterial scaffold can be slid onto the column such that the column provides internal stability to the thin fiber of the scaffold material.

In some embodiments, the gear-ring with inset is configured to be snapped onto the uncapped end of the column.

In some embodiments, provided is a method of using the 3D column microdevice as described herein for seeding an exterior of a biodegradable polymer conduit with cells and for filling an interior luminal space of the conduit with macromolecules, growth factors, adhesion molecules, and/or cells.

In some embodiments, the interior luminal space may be the same or different composition than the exterior surface of a biodegradable polymer conduit.

In one aspect, this disclosure provides a rectangular microdevice for use in seeding of cells onto fibers comprising a rectangular body having inset recesses on one side of the body and a rectangular opening on the other side of the body.

In some embodiments, the body is made from polylactic acid (PLA), polycaprolactone (PCL), polyvinyl alcohol (PVA), or any combination thereof. In some embodiments, the fibers are selected from the group comprising resorbable fibers, poly(lactic-co-glycolic acid) (PLGA) derived fibers, polyglycolic acid (PGA) derived fibers, a tyrosine-derived polymer with a high concentration of poly(ethylene glycol) (PEG), and any combination thereof.

In some embodiments, the rectangular microdevice as described herein has a weight of about 1 gram to about 10 grams.

In some embodiments, provided is a method of using the rectangular microdevice as described herein for real-time microscopic imaging of cells attaching to fibers in vitro.

### BRIEF DESCRIPTION OF THE DRAWINGS

FIG. 1 illustrates the oblique frontal view of a large snap-fit fixture. A indicates inset depression; B indicates outwardly protruding tabs; C indicates openings.

FIG. 2 illustrates the oblique bottom view of a large snap-fit fixture. D indicates holes; F indicates lip of the barrel opening.

FIG. 3 depicts the oblique frontal view of a small snap-fit fixture. E indicates small snap-fit fixture is snapped in by means of the projection.

FIG. 4 depicts the oblique bottom view of a small snap-fit fixture. G indicates hollow of the barrel.

FIG. 5 shows the oblique frontal and top view of a hollow column with flange. The 3D column microdevice works such that the center column A is inserted through the central hole in the gear and the ring is slid down to the flange on the column B.

FIG. 6 shows the oblique frontal view and top view of a gear with hole. C indicates the tabs of the gears. D indicates the hollow of the gear, allow for insertion of the hollow column with flange.

FIG. 7 depicts the oblique frontal view and top view of a gear with depression/end cap. E indicates solid inner base of “Gear with Depression”; this gear serves as a removable end cap for the fully assembled device in FIG. 9, first panel (outer layer of conduit cell seeding).

FIG. 8 illustrates the oblique frontal view and top view of a hollow barrel. F is the lip of the barrel opening. G is the hollow of the barrel.

FIG. 9 is a fully assembled device.

FIG. 10 shows a fiber fixture for cell seeding. A indicates depressions in which the ends of fibers can be set using liquid or other adhesive (i.e., glue or mechanical fixture). B indicates hollow of rectangular fiber fixture, across which synthetic or natural fibers can be stretched and secured. These fibers can then be coated with cells, or alternatively coated with hydrogel or other coatings including poly-d-lysine or other charged coatings to facilitate cell adhesion.

FIGS. 11A-11F. Fiber morphology assessed using a scanning electron microscope (SEM). A: SEM image of unaligned E1001(k) biopolymer fibers forming flat electrospun sheet, at 500× magnification; B: 5000× magnification; C: 8-mm circular sections of the polymer; D: fully assembled configuration; E: the assembly; F: the assembly of FIG. 11E placed longitudinally into a 24-well plate.

FIG. 12 is 10× magnification imaging of trat Schwann cells growing on E1001k fibers.

### DETAILED DESCRIPTION OF THE DISCLOSURE

Disclosed herein is a microdevice and methods of using the microdevices to grow and seed cells onto various biocompatible materials such that those cells can later be successfully engrafted into an animal, e.g., a human host.

A microdevice, also referred to as a snap-fit fixture, includes two interlocking pieces (e.g., a large snap-fit fixture and a small snap-fit fixture) which connect to create a platform on which to place a biocompatible polymer material that can be seeded on one or both sides with cells of similar or different tissue identities. The microdevice may be used to create biomaterial scaffolds of multiple layers of polymer sheets and cells. These cells may be selected from: nerve cells, including, but not limited to, neurons and their support cells such as Schwann cells, oligodendrocytes, astrocytes, microglia, and other neural-specific support cells, such as peripheral nerve fibroblasts; connective tissue cells including, but not limited to, cartilage cells, osteocytes, muscle cells (skeletal muscle, smooth muscle, cardiac muscle), fibroblasts, and myofibroblasts; endothelial cells and other cells that support circulatory system vasculature such as pericytes; gastrointestinal cells such as columnar and squamous epithelial cells, goblet cells, parietal cells, chief cells, cholangiocytes, pancreatic islet cells and acinar cells, hepatocytes, or stromal cells; and genitourinary tract cells including, but not limited to, cuboidal and transitional epithelial cells of the urinary tract, simple columnar epithelium of the fallopian tubes, and complex epithelial cells of the seminiferous tubules including Sertoli and Leydig cells.

The biodegradable polymer may be solution-processed or melt processed biodegradable polymer in the form of a tube, or a flat sheet by molding, extrusion, electrospinning, braiding or weaving or any other known fabrication method. The biocompatible polymer material may be an electrospun biodegradable polymer in the form of a tube or a flat sheet.

The flat sheet may be dried, and refrigerated. Shortly prior to culture experiments, the flat sheet of biodegradable polymer may be cut, e.g., into about 5 mm to about 12 mm, or about 8-mm circular section, as needed. The cut polymer sheet may be secured into the snap-fit fixture after sterilizing all components, e.g., under UV light for 30 minutes.

The biocompatible polymer material may be constructed from a tyrosine-derived or tyrosol-derived polymer, which may alternatively be referred to as a tyrosine-polymer, or tyrosol-derived polymer, respectively. The tyrosine-derived or tyrosol-derived polymer have non-inflammatory degradation bioproducts. The conduit may be composed of a tyrosine-derived polymer, for example, desaminotyrosyl-tyrosine ethyl ester (DTE), desaminotyrosyl-tyrosine (DT), or a combination thereof. The conduit may be composed of desaminotyrosyl-tyrosine ethyl ester (DTE), desaminotyrosyl-tyrosine (DT), and polyethylene glycol (PEG). The molar fraction of free carboxylic acid units and PEG units in the polymer described herein can be adjusted to modify the mechanical properties and degradation rates of NADs made from such polymers. For example, polymers with lower amounts of free carboxylic acid will tend to have longer lifetimes in the body. Further, by otherwise adjusting the amount of free carboxylic acid in the polymers across the range of preferred molar fraction, the resulting polymers can be adapted for use in various applications requiring different device lifetimes. In general, the higher the molar fraction of free carboxylic acid units, the shorter the lifetime of the device in the body and more suitable such devices are for applications wherein shorter lifetimes are desirable or required.

The biocompatible polymer material may be composed of a tyrosol-derived polymer, for example, U.S. Publication No. 2020/0181321 and WO 2021/055090, which are incorporated by reference herein in their entirety. The conduit may be composed of poly(HTy glutarate), poly(HTy suberate), poly(HTY dodecanedioate), poly(HTy phenylenediacetate), or any combination thereof. The conduit may be composed of poly(HTy glutarate).

The biocompatible polymer material may be constructed from a biodegradable polymer having repeating units of the structure (Formula I):

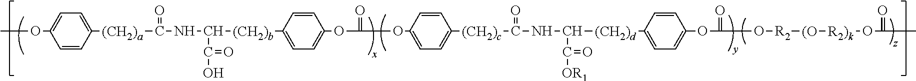

wherein a and b are independently 0 or an integer between 1 and 6, inclusive; wherein c and d are independently 0 or an integer between 1 and 6, inclusive; wherein each R₁ is independently selected from the group consisting of straight and branched alkyl groups containing up to 18 carbon atoms; wherein each R₂ is independently an alkylene group containing up to 6 carbon atoms; wherein k is between about 20 and about 200; and wherein x ranges between about 0.02 and about 0.20; z ranges between about 0.005 and 0.10; and x+y+z=1.00. In some embodiments, a and b are two and one, respectively. In some embodiments, c and d are two and one, respectively, and R₁ is ethyl. In some embodiments, R₂ for said polymer is ethylene and k is between about 25 and about 50.

The synthesis of various polycarbonate polymers is generally known in the art, including for example, the methods disclosed in U.S. Pat. Nos. 6,120,491 and 6,475,477, the disclosures of which are incorporated herein by reference. The polyacrylates and other polycarbonates disclosed in U.S. Pat. No. 6,120,491 are also incorporated herein by reference for use in construction of the polymer conduits herein. Polymers having pendent free carboxylic acid groups are preferably prepared from the corresponding benzyl and tert-butyl ester polymers to avoid cross-reaction of the free carboxylic acid group with co-monomers. The benzyl ester polymers may be converted to the corresponding free carboxylic acid polymers by the palladium catalyzed hydrogenolysis method disclosed in U.S. Pat. No. 6,120,491. The tert-butyl ester polymers may be converted to the corresponding free carboxylic acid polymers through the selective removal of the tert-butyl groups by the acidolysis method disclosed in U.S. Patent Publication No. 20060034769, also incorporated herein by reference.

Polymers may be selected which degrade or resorb within a predetermined time. For this reason, embodiments may include polymers with molar fractions of monomeric repeating units with pendant fee carboxylic acid groups, such as DT, between about 2 and about 20 mol %, and preferably between about 5 and about 20 mol %.

Poly(alkylene glycol) segments, such as PEG, decrease the surface adhesion of the polymers. By varying the molar fraction of poly(alkylene glycol) segments in the block copolymers provided by the present invention, the hydrophilic/hydrophobic ratios of the polymers can be changed to adjust the ability of the polymer coatings to modify cellular behavior. Increasing levels of poly(alkylene glycol) inhibit cellular attachment, migration and proliferation. Secondarily, PEG increases the water uptake, and thus increases the rate of degradation of the polymer. Accordingly, in an embodiment, polymers are selected in which the amount of poly(alkylene glycol) is limited to between 0.5 and about 10 mol %, and preferably between about 0.5 and about 5 mol %, and more preferably between about 0.5 and about 1 mol %. The poly(alkylene glycol) may have a molecular weight of 1 k to 2 k.

The biodegradable polymer may be selected having intrinsic physical properties appropriate for use in polymer conduits with suitable mechanical properties including elasticity, rigidity, strength and degradation behavior. Such polymers include, if the polymer is amorphous, polymers with a glass transition temperature greater than 37 °C when fully hydrated under physiological conditions and, if the polymer is crystalline, a crystalline melting temperature greater than 37 °C when fully hydrated under physiological conditions.

It is to be understood that other biodegradable and biocompatible polymers can be used to form fibers that provide or reinforce certain desirable properties of the resulting polymer conduits. Examples of other polymers that may be used include, but are not limited to, poly(lactic acid), poly(glycolic acid), poly(lactic-co-glycolic acid), polycaprolactone, various poly(amino acid)s and polyanhydrides. Other natural or non-natural fiber materials, for example, collagen, cellulose, chitosan, and their derivatives, may alternatively or additionally be utilized to provide or reinforce certain desirable properties of the resulting polymer conduits (see, for example, U.S. Pat. No. 8,216,602).

The biodegradable polymer may be a biodegradable polymer conduit and may be constructed from any polymer disclosed in U.S. Patent Publication No. 2018/0280567, which is incorporated by reference herein in its entirety.

The biodegradable polymer may comprise a recurring unit of Formula XVIII:

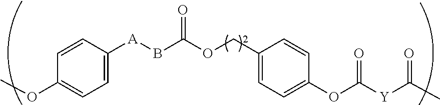

wherein:
(a) A is CH₂ or CH₂CH₂, B is a bond, Y is selected from the group consisting of (CH₂)₂, (CH₂)₃, CH₂OCH₂, (CH₂)₄, CH₂CH═CHCH₂, (CH₂)₅, (CH₂)₆, and (CH₂)₁₀;
or
(b) A is CH₂CH₂, B is selected from the group consisting of —O—CO—CH₂CH₂, O—CO—CH₂CH₂CH₂, and —O—CO—CH₂OCH₂ and bonded to A via oxygen, Y is selected from the group consisting of (CH₂)₂, (CH₂)₃, CH₂OCH₂, (CH₂)₄, CH₂CH═CHCH₂, (CH₂)₅, (CH₂)₆, and (CH₂)₁₀, or any other polymer disclosed in U.S. Patent Publication No. 2020/0181321, which is incorporated by reference herein in its entirety. The biodegradable polymer conduit may be constructed from PEG block polymers of the foregoing polymer.

The biodegradable polymer conduit may be constructed from a biocompatible polymer comprising a plurality of units of Formula I,

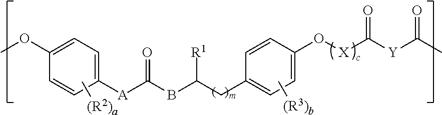

wherein:
A is a bond, C₁₋₃alkylene, OC₁₋₃alkylene, CH═CH, C₁₋₃alkylene-O—CO—C₂₋₅alkylene, C₁₋₃alkylene-O—CO—C₁₋₂alkylene-O—C₁₋₂alkylene;
B is oxygen, or NR^{a} wherein R^{a} is H or an optionally substituted C₁₋₁₀ alkyl, or —O-E-OCO— wherein E is selected from the group consisting of C₁₋₃₀ alkylene, C₂₋₃₀ alkelene, C₁₋₃₀ alkynylene, C₁₋₃₀ heteroalkylene, C₂₋₃₀ heteroalkelene, C₁₋₃₀ heteroalkynylene, C₆₋₃₀ arylene, C₇₋₃₀ alkylarylene, C₈₋₃₀ alkenlarylene, C₈₋₃₀ alkynylarylene, and C₂₋₃₀ heteroarylene,
R¹ is H or COOR wherein R^{b} is selected from the group consisting of H, C₁₋₁₀ alkyl and C₁₋₁₀ alkylaryl containing up to 18 carbon atoms;
R² and R³ are independently halogen, C₁₋₄alkyl or OC₁₋₄alkyl;
X in each instance is an amino acid moiety represented as

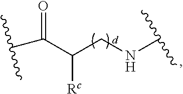

wherein
R^{c} is H, or an unsubstituted or substituted group selected from the group consisting of C₁₋₆ alkyl, C₁₋₃ alkylene-S—C₁₋₄ alkyl, C₁₋₃ alkylene-aryl, C₁₋₃ alkylene-heteroaryl, C₁₋₆ alkylene-COOH, C₁₋₆ alkylene-N(R′)₂, and C₁₋₆ alkylene-CON(R′)₂, wherein each Rₓ is independently H or C₁₋₆ alkyl;
Y is an unsubstituted or substituted group selected from the group consisting of C₁₋₁₀ alkylene, C₆₋₁₀arylene, C₁₋₃ alkylene-O—C₁₋₃alkylene, O—C₁₋₆alkylene, C₁₋₃alkylene-S—C₁₋₃-alkylene, C₁₋₃alkylene-CH═CH—C₁₋₃alkylene, C₃₋₈cycloalkylene, C₁₋₃ alkylene-C₄₋₈cycloalkylene-C₁₋₃alkylene, C₁₋₃ alkylene-C₆₋₁₀arylene-C₁₋₃-alkylene, C₁₋₆alkylene-NR^{c}—, C₁₋₃ alkylene-CH(NHBoc)- and C₁₋₃ alkylene-NR^{c}—C₁₋₃ alkylene, wherein R^{e} is selected from the group consisting of H, C₁₋₆ alkyl, C₆₋₁₀ aryl, C₁₋₃ alkylene-C₆₋₁₀aryl and CON(R^{f})₂, wherein each R^{f} is independently H or C₁₋₆ alkyl;
a and b are independently an integer ranging from 0 to 4;
c is an integer ranging from 1 to 8; d is 0 or 1; and m is an integer ranging from 1-3. In some embodiments, A is C₁₋₃alkylene, C₁₋₃alkylene-O—CO—C₂₋₅alkylene, or C₁₋₃alkylene-O—CO—C₁₋₂alkylene-O—C₁₋₂alkylene. In some embodiments, A is CH₂ or CH₂CH₂. In some embodiments, R¹ is H. In some embodiments, the amino acid moiety is derived from natural amino acid. In some embodiments, the amino acid moiety is derived from essential amino acid selected from the group consisting of phenylalanine, valine, threonine, tryptophan, methionine, leucine, isoleucine, lysine, and histidine. In some embodiments, Y is selected from the group consisting of C₁₋₅alkylene, phenylene, and C₁₋₂ alkylene-O—C₁₋₂alkylene. In some embodiments, R² and R³ in each occurrence are independently a bromine or iodine; a and b are independently 0, 1 or 2.

In some embodiments, the biocompatible polymer further includes a recurring unit of the formula II-a:

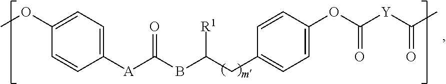

wherein m′ is an integer ranging from 1-3.

In some embodiments, the biocompatible polymer further includes a recurring unit of the formula II-b:

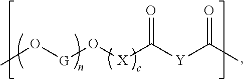

wherein G is C₂₋₃-alkylene, n is an integer ranging from 4 to 3000.

In some embodiments, the biocompatible polymer further includes a recurring unit of the formula II-c:

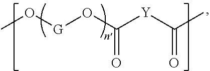

wherein G is C₂₋₃-alkylene, n′ is an integer ranging from 4 to 3000.

In some embodiments, the biocompatible polymer further includes a copolymer unit selected from the group consisting of poly(ethylene glycol), polycaprolactone-diol, polycaprolactone, poly(trimethylene carbonate), polylactide, polyglycolide, and poly(lactic-co-glycolic acid).

In some embodiments, the biocompatible polymer further includes a structure of Formula I-b,

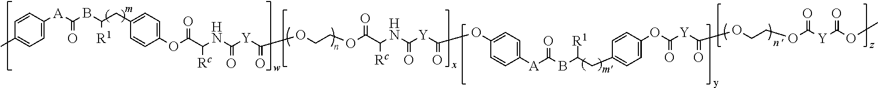

wherein w ranges from about 0.001 to 1, x ranges from 0 to 0.999, y ranges from 0 to 0.999, z ranges from 0 to 0.999, and w+x+y+z=1.000. In some embodiments, A is selected from the group consisting of C₁₋₃ alkylene, C₁₋₃ alkylene-O—CO—CH₂CH₂, C₁₋₃ alkylene-O—CO—CH₂CH₂CH₂, and C₁₋₃ alkylene-O—CO—CH₂OCH₂; B is oxygen. In some embodiments, Y is selected from the group consisting of (CH₂)₂, (CH₂)₃, CH₂OCH₂, (CH₂)₄, CH₂CH═CHCH₂, (CH₂)₅, (CH₂)₆, and (CH₂)₁₀. In some embodiments, R¹ and R^{c} are H.

The biocompatible polymer material may be constructed from PEG block polymers of any of the foregoing biocompatible polymers.

Also incorporated herein by reference in entirety are: U.S. Pat. No. 5,099,060, in particular, for its disclosure related to polycarbonate synthesis; U.S. Pat. No. 5,216,115, in particular, for its disclosure related to polyarylate synthesis; U.S. Pat. No. 6,048,521, in particular, for its disclosure related to PEG block copolymerization; and U.S. Patent Publication No. 2006/0034769, in particular, for its disclosure related to the synthesis of polymers with free acid groups.

The biocompatible polymer material may be multilayered. It may include one layer, two layers, three layers, or four layers. Each layer may contain aligned (which may also be referred to as oriented) or unaligned (which may also be referred to as non-oriented) biopolymer fibers.

The snap-fit fixture may include a large snap-fit fixture and a small snap-fit fixture, which reversible interlock. The snap-fit fixture may be made from polylactic acid (PLA), polycaprolactone (PCL), polyvinyl alcohol (PVA), or in any combination thereof, or other commonly used polymers for 3-D printing, extrusion, photolithography, or other known polymer processing techniques.

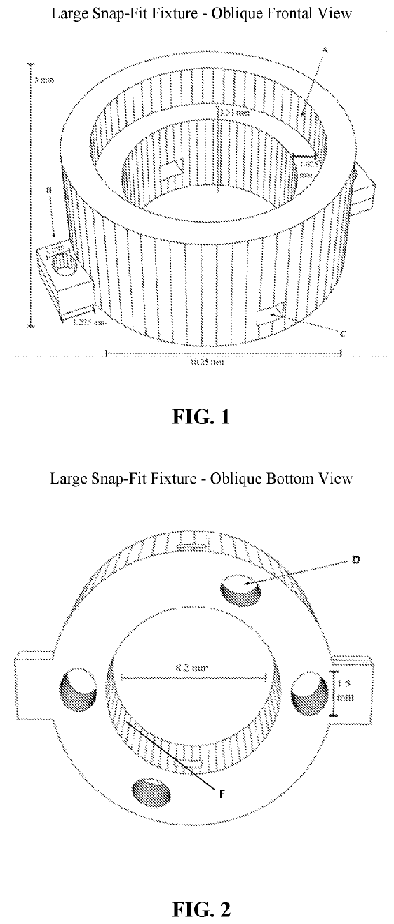

The large snap-fit fixture and the small snap-fit fixture connect by snapping together such that they make one interlocking device. The large snap-fit fixture may be about 2 mm to about 20 mm, or about 3 mm to about 10 mm in height, and about 5 mm to about 40 mm, about 5 mm to about 25 mm, or about 5 mm to about 15 mm in diameter (not including the tabs (B in FIG. 1). The aperture (F in FIG. 2) in the center of the large snap-fit fixture may be about 3 mm to about 30 mm, about 3 mm to about 20 mm, or about 3 mm to about 12 mm in diameter. The small snap-fit fixture may be about 2 mm to about 20 mm, or about 3 mm to about 10 mm in height, and about 5 mm to about 40 mm, about 5 mm to about 25 mm, or about 5 mm to about 15 mm in diameter (not including the tabs (B in FIG. 1)). The aperture (G in FIG. 4) in the center of the small snap-fit fixture may be about 3 mm to about 30 mm, about 3 mm to about 20 mm, or about 3 mm to about 12 mm in diameter. The aperture in the small snap-fit fixture may be smaller in diameter than the aperture in the large snap-fit fixture. The large snap-fit fixture and the small snap-fit fixture may facilitate the deposition of proteins and other macromolecules, which may further facilitate the adhesion and growth of cells onto one or both of the snap-fit fixtures and/or biocompatible polymers.

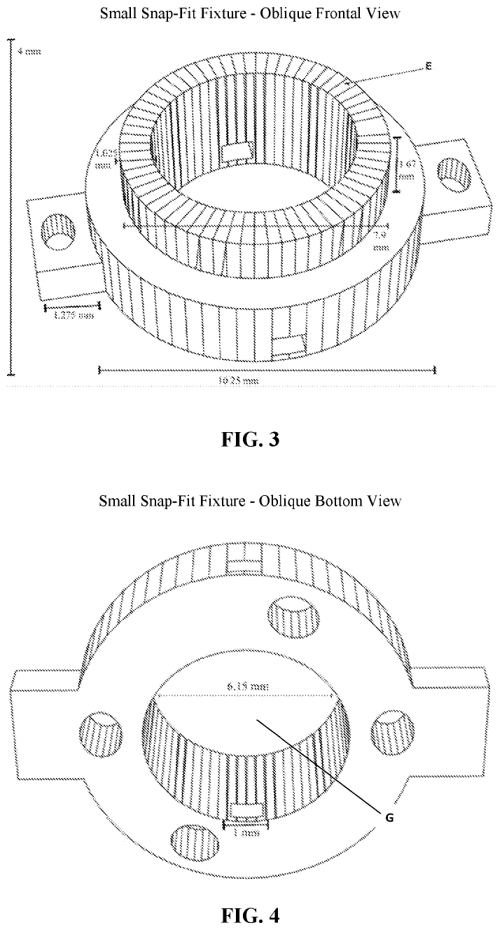

FIGS. 1 and 2 are drawings of a large snap-fit fixture. FIGS. 3 and 4 are drawings of a small snap-fit fixture. Referring now to FIGS. 1-4, in the inset depression marked (A) a piece of polymer fiber or biomaterial sheet can be seated such that when the small snap-fit fixture is snapped in by means of the projection (E), the polymer fiber is sandwiched between the two fixtures that make up the snap-fit fixture. The weight of the snap-fit fixture is such that it can then be immersed in cell culture fluid, water, or other media within in a cell culture dish, well, or other container, and the device will remain submerged, or at the bottom, of the dish, well or container. The weight may be from about 0.25 grams to about 5 grams, or about 2 grams. When interlocked together, the snap-fit fixture allows for one well to be formed on either side of the inset location of the biomaterial sheet, creating two wells. A first solution containing cells may be pipetted or otherwise placed onto one side of the biomaterial sheet, and allowed to interact and adhere for about 30 minutes to about 7 days. The snap-fit fixture may be flipped 180 degrees along its transverse axis, such that the opposing side of the biomaterial sheet is facing upward, and a second solution containing cells, being the same or different from the first solution, and optionally containing a different type of cells, may be pipetted or otherwise placed to this opposing side of the biomaterial sheet such that the same or different cells and/or molecules can be applied to this opposing side of the biomaterial sheet. This may then be allowed to interact and adhere for about 30 minutes to about 7 days.

The snap-fit fixture may be modified to accommodate multiple layers of biomaterial sheet or polymer fiber by repeating a process such as described above. After the steps disclosed above to seed cells on two opposing sides of a biomaterial sheet, the snap-fit fixture may be opened (such the interlock detached), and another layer of biomaterial sheet may be inserted, the snap-fit fixture snapped in place to interlock and additional cells pipetted and then seeded onto the newly-placed biomaterial sheet layers. The snap-fit fixture may optionally be opened and the biomaterial removed for morphological and/or histological analysis. While manipulating the snap-fit fixture, outwardly protruding tabs (B), optionally placed on opposite sides of each of the large snap-fit fixture and small snap-fit fixture, are provided such that they can be grasped by tweezers, forceps, or other tools to enable easier manipulation of the fixture. The outwardly protruding tabs also have insets of approximately 1 mm diameter and depth such that the prongs of tweezer or other tools can be inserted to facilitate the manipulation by turning or rotation of the entire assembly or its respective halves to facilitate movement or separation of the whole or halves, respectively. Holes (D) in the large snap-fit fixture, shown in FIG. 2, provide a similar purpose on the bottom of each half, such that tools can be inserted into the depressions to aid in the removal or movement of the device halves. Openings (C) in the large snap-fit fixture, shown in FIG. 1, are open holes that extend from the outside of the fixture into the interior space such that culture media or other fluids can flow from the external surroundings into the snap-fit fixture when it is submerged in fluid. This allows for transit of nutrients in and out of the snap-fit fixture.

A second microdevice, also referred to as a 3D column microdevice, is disclosed for use in seeding the exterior of a biodegradable polymer conduit with cells and for filling the interior luminal space with macromolecules, growth factors, adhesion molecules, and/or cells, such that the interior luminal space may be the same or different composition than the exterior surface of a biodegradable polymer conduit. In this way, the interior and exterior surfaces of a biodegradable polymer conduit may have different tissue compositions. The biodegradable polymer conduit may be one made from any of the biodegradable polymers described herein, or otherwise disclosed in International Application No. PCT/US2022/077140, title: “CELLULARIZED NERVE REGENERATION GRAFT AND METHODS OF MAKING THE SAME,” co-owned and concurrently filed on Sep. 28, 2022, the contents of which are incorporated herein in its entirety.

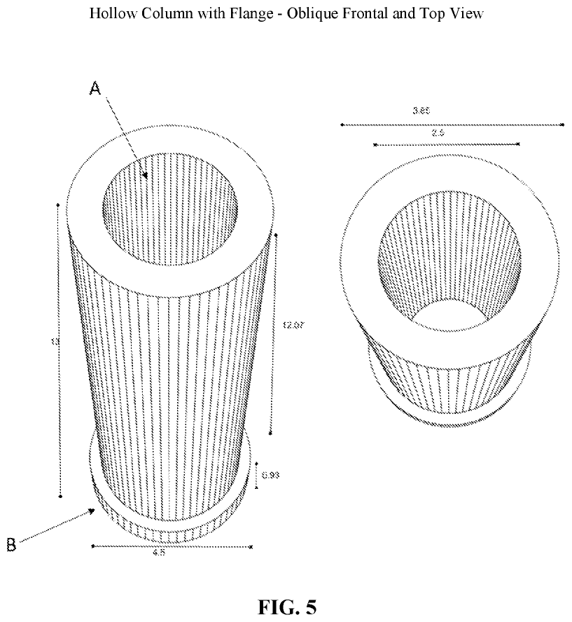

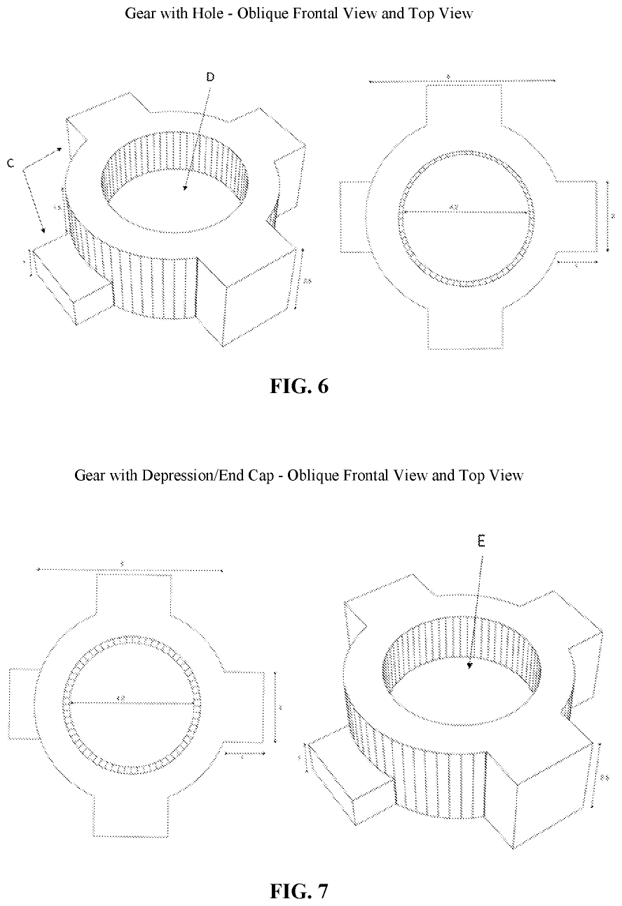

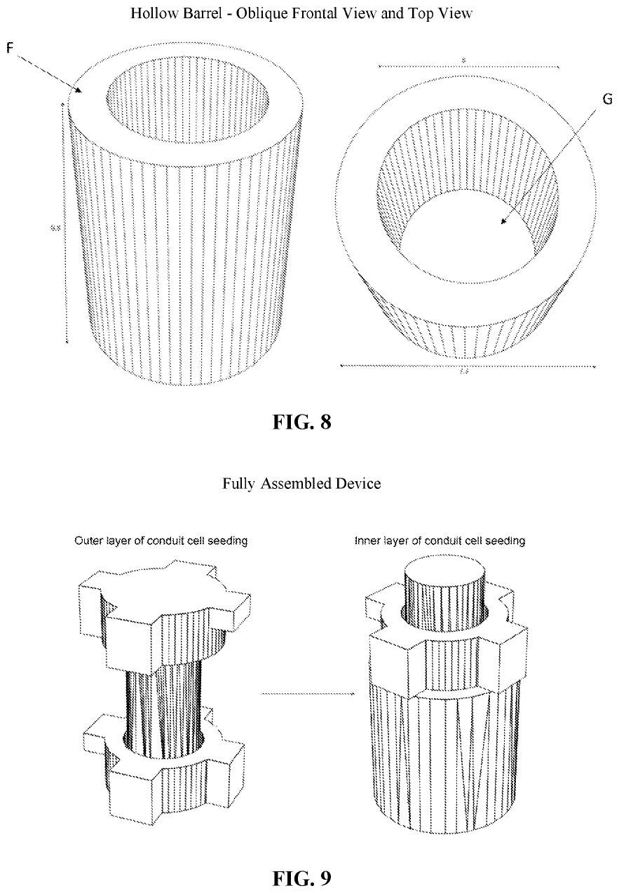

The 3D column microdevice consists of multiple pieces: a barrel (see, e.g., FIG. 8), a center column (see, e.g., FIG. 5), a gear-shaped ring with a central depression inset (see, e.g., FIG. 7), and a gear-shaped ring with a patent opening (see, e.g., FIG. 6). The 3D column microdevice works such that the center column is inserted through the central hole in the gear and the ring is slid down to the flange on the column B in FIG. 5. Then, a tubular biomaterial scaffold can be slid onto the column such that the column provides internal stability to the thin fiber of the scaffold material. The gear-ring with inset is then snapped onto the uncapped end of the column. This device can then be placed into a culture dish onto its longitudinal axis and seeded with cell suspension or molecular factors. To do so, the device is rotated by means of turning the tabs of the gears C in FIG. 6. One tab on each gear is colored differently and is shorter in height than the remainder of the tabs of the device, such that it facilitates visualization of the starting and ending point during cell seeding. The biodegradable polymer conduit is seeded with cell suspension on one arc-length of the conduit, the cells are allowed to adhere, and then the device is rotated 90 degrees along its longitudinal axis, exposing a second arc of the conduit. Cell suspension or molecular factors are then applied in a similar manner, and the process may be repeated until the entire circumference of the biodegradable polymer conduit is seeded with cell suspension or molecular factors, as needed. The entire 3D column microdevice can then be submerged in cell culture media or other fluid, as needed, and cells can be grown to confluence, or to the desired proliferation end point. The 3D column microdevice may then be stood upright on the flanged end of the column B in FIG. 5 and the gear-ring with inset can be removed from the end of the column. The column can then be placed with its flange resting in the hollow top of the barrel with the gear-ring resting on the lip of the barrel opening F in FIG. 8. A tool, such as a tweezer or other instrument, can be inserted into the patent end of the column A in FIG. 5 and the column is pushed into the hollow of the barrel G in FIG. 8 such that the column retracts from its place of support within the interior of the biodegradable polymer conduit, thus leaving a biomaterial tube with cells or molecular factors on its exterior and the interior open, resting on top of the open gear-ring and the entire retracted assembly. A filler can be inserted the interior luminal space of the biodegradable polymer conduit. This filler may comprise a hydrogel matrix, augmented hydrogel matrix, cells, fibers, or other materials or combinations thereof to support the interior lumen and to provide appropriate tissue morphology and function for the interior.

The hydrogel matrix may be defined as a cross-linked hydrophilic polymer that does not dissolve in water and is capable of absorbing large quantities of water or other biological fluids, which may be made from RADA-16 peptides, collagen, gelatin, alginate, hyaluronic acid, or any other known hydrogel materials. The augmented hydrogel matrix is a hydrogel matrix that is combined via blending, mixing, chemical conjugation, or other known method, with another biochemical factor, such as a growth factor, guidance cue, structural peptide, adhesion factor, other chemical agent, or combination thereof.

The augmented hydrogel matrix may be a RADA16 peptide, collagen, gelatin, alginate, or hyaluronic acid hydrogel. The augmented hydrogel matrix may be functionalized with a growth factor to support cell growth, either by physical mixing with the hydrogel matrix or by chemical conjugation with the hydrogel matrix, or any combination thereof. The growth factor may be selected from Neuregulin 1 (NRG1), EGF, FGF, NGF, PDGF, VEGF, IGF, GMCSF, GCSF, TGF, Erythropoietin (EPO), TPO, BMP, HGF, GDF, Neurotrophins (e.g., GDNF, CNTF, BDNF, NT3), netrins, MSF, SGF, or any combination thereof.

The augmented hydrogel matrix may include one or more additives selected from a basal medium known for use in supporting the growth of cells (e.g., Dulbecco's Modified Eagle Medium (DMEM), fetal bovine serum (FBS), an antibiotic (e.g., penicillin, streptomycin or a combination thereof), forskolin, and any combination thereof.

In this way, the biodegradable polymer conduit may be developed into a number of different human or animal tissues including nerves (central or peripheral), vasculature such as arteries, veins, arterioles, venules, capillaries, or other circulatory structures, lymphatic vessels, digestive structures including the esophagus, portions of the small and large bowels, and other structure such as bone. Additionally, the 3D column microdevice allows for the attachment of flow reactors or other devices to promote continuous movement of nutrients into proximity of the biodegradable polymer conduit and seeded cell and/or molecular components. The 3D column microdevice can also allow for the attachment of electrical leads to provide pulsatile force to developing muscle fibers, neurons, or other cells and tissues, or to stabilize the biodegradable polymer conduit to receive magnetic, optical, or sonographic stimulation. Once the biodegradable polymer conduit is stable, it can be completely removed from the 3D column microdevice and placed into other conditions or onto an accessory device.

The 3D column microdevice may also be used to seed the system in the interior luminal space of the biodegradable polymer conduit. The 3D column microdevice may be the same microdevice used for seeding fibroblasts.

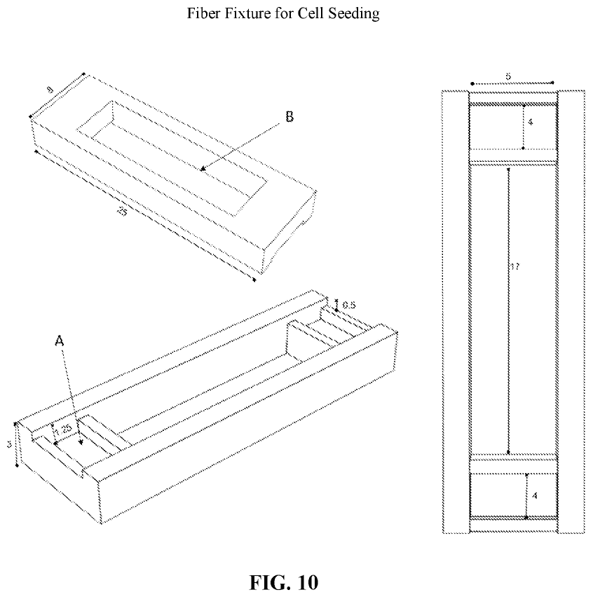

A third microdevice, also referred to as a rectangular microdevice, that facilitates the seeding of cells onto fibers is disclosed. These fibers can be solid, hollow, braided, and can be made of many different materials. The fibers may be resorbable fibers, including but not limited to, fibers may be made from poly(lactic-co-glycolic acid) (PLGA), polyglycolic acid (PGA), a tyrosine-derived polymer with a high concentration of poly(ethylene glycol) (PEG), or any combination thereof. In this rectangular microdevice, fibers are stretched across the recessed portion of the device. The fibers may be glued or clamped into the inset recesses A in FIG. 10. The device may be turned over, such that the fiber side is facing downward. When this action is performed, the top of the device reveals an opening B in FIG. 10 through which molecular factors can be applied by pipette or otherwise to the fibers. The rectangular microdevice may be placed into a container such as a cell culture dish and molecular factors may be applied via pipet or other tool and the molecular factors may be allowed to adhere over a period of time, e.g., hours to about 7 days. Once the fibers are coated, a cell suspension may be applied to the fibers and the entire device may be submerged in cell culture media or other fluid. The rectangular microdevice is weighted such that it will stay submerged in cell culture media, water, or other common low viscosity fluids. The weight of the microdevice may be about 1 gram to about 10 grams, or about 5 grams. Cells may be grown to confluence or to the desired point, and the fibers may be detached from the device for subsequent use.

The bottom side of the rectangular microdevice may be modified to make a rounded inset depression on either end, and a bundle of fibers can be placed in a similar manner to the single layer of fibers. The fiber bundle may be seeded in a similar manner to the single layer of fibers with molecular factors and/or cells, and the bundle can be used to generate the interior structure of a tube or other structure.

The rectangular microdevice may have a rectangular opening or indent on one side or the microdevice that does not extend through the entire device. On the opposing side of the rectangular microdevice are two recesses at opposing ends for holding fibers in place. The rectangular microdevice may be about 10 mm to about 50 mm long, about 5 mm to about 25 mm wide, and/or about 2 mm to about 5 mm in height (as to reduce the total volume of cell culture media used). The recessed area for the placement of fibers may be about 0.2 mm to about 0.5 mm deep from the surface of the microdevice as to facilitate close proximity to the bottom of a culture dish when the device is turned over. This last aspect allows for real-time microscopic imaging of cells attaching to fibers in vitro.

The rectangular microdevice may be made from polylactic acid (PLA), polycaprolactone (PCL), polyvinyl alcohol (PVA), or in any combination thereof, or other commonly used polymers for 3-D printing, extrusion, photolithography, or other known polymer processing techniques.

### EXAMPLES

### Example 1

Manufacturing of biopolymer and assemblies for Cellularized Nerve Regeneration Graft as disclosed in International Application No. PCT/US2022/077140, title: “CELLULARIZED NERVE REGENERATION GRAFT AND METHODS OF MAKING THE SAME,” co-owned and concurrently filed on Sep. 28, 2022, is incorporated by reference herein as an example of how the 3D column microdevice and the snap-fit fixture may be used.

Flat sheets were made using a large diameter (5 cm) mandrel that was laterally oscillated to obtain 13×21 cm mats. The speed of the mandrel was controlled by a DC power supply (Model 1627A, BK Precision, Yorba Linda, CA). The linear speed was set at 30 meters per minute (mpm) for unaligned layers and 650 mpm for aligned layers.

Multi-layered scaffolds were prepared in three steps: 16% polymer solution was spun into an unaligned layer at 2 mL/h for 30 minutes, followed by an additional unaligned layer with 10% solution at 1 mL/h for 30 minutes, and finally an aligned layer with 10% solution at 1 mL/h for 30 minutes.

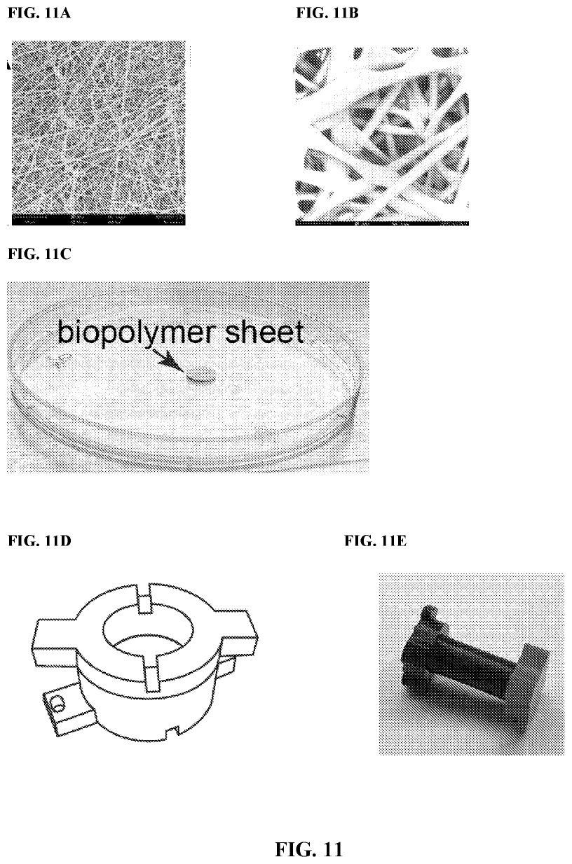

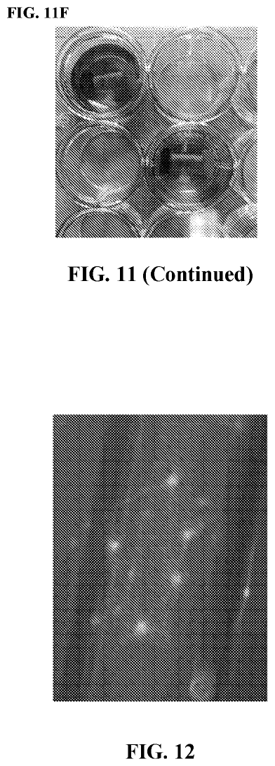

Initially, these multilayer scaffolds were electrospun as flat sheets, dried slowly at RT (Room Temperature), and refrigerated at 4 °C until needed. Shortly prior to culture experiments, the polymer was cut into 8-mm circular sections (as shown in FIG. 11C), as needed, using an 8-mm diameter steel hollow punch. Cut scaffolds were secured into the snap-fit fixture shown in FIGS. 1-4 after sterilizing all components under UV light for 30 minutes.

Following proof of cell compatibility with the flat biopolymer sheets, hollow biopolymer conduits were then prepared using smaller diameter (1.5, 2, and 4 mm) mandrels. The mandrels were coated with PEG gel to facilitate the release of the conduit after electrospinning. These mandrels were mounted onto a chuck (IKA, model R20DS1) and spun at approximately 200 rpm for 10 min to 2 h to obtain tubes of different wall thicknesses and tube diameters. After electrospinning, the mandrel was removed from the chuck, wetted slightly with deionized water, and the polymer conduit was carefully removed by sliding it off the mandrel. Conduits were allowed to dry slowly and then refrigerated at 4 °C, until needed, to prevent decomposition. For culture experiments, shortly prior to cell seeding the conduits were cut into 5-mm length sections using stainless steel surgical scissors and then sterilized under UV light for 30 minutes. Scaffold thickness was measured using a micrometer. Fiber morphology was assessed using a scanning electron microscope (SEM) (Phenom ProX, Nanoscience Instruments, Phoenix, AZ).

SEM image of unaligned E1001(k) biopolymer fibers forming flat electrospun sheet, at 500× magnification is shown in FIG. 11A. SEM image of the same electrospun sheet in FIG. 11A but at 5000× magnification is shown in FIG. 11B. FIGS. 1 and 2 show a bottom half of 3D-printed snap fixture for holding flat biopolymer sheet. An 8-mm diameter piece of electrospun E1001(k) scaffold may be placed into the round inset shown within the device. FIGS. 3 and 4 show a top half of 3D-printed snap fixture. Once the electrospun biodegradable polymer sheet is placed into the bottom half piece, the top half piece is snapped in, thus securing an electrospun biopolymer sheet in place. This fully assembled configuration is shown in FIG. 11D. Once the device is fully assembled, cell suspension can be pipetted into the wells created on either side of the snap fixture. This allows cells to adhere to only one side of the scaffold fibers while preventing cell migration to the other side. The device is sized with an outer width (from tab to tab) of about 12 mm such that the entire device fits into, and can be turned inside, a well of a standard 24-well culture dish. Holes through the side walls allow for culture media to flow through the device. Indentations in the tabs allow for handling of the device using forceps. FIG. 11C is a sample circular 8-mm DTE biopolymer sheet.

To seed cells onto the exterior and interior of the electrospun biodegradable polymer conduit, also referred to herein as an electrospun biopolymer tube or an electrospun biodegradable polymer conduit, a device was assembled such that: a rod was inserted through hollow gear. The electrospun biopolymer tube was then placed over the rod and capped with solid gear, resulting in an assembly, with the electrospun biopolymer tube here shown in transparent shading. The assembly (FIG. 11E) was then placed longitudinally into a 24-well plate (shown in FIG. 11F) and seeded with 40 uL of fibroblast suspension at a density of 2.5×10⁵ cells/mL by pipetting the volume across the length of the exterior of the exposed electrospun biopolymer tube. The cells were allowed to attach to the scaffold for 2 minutes and then the device was rotated about its longitudinal axis by 90 degrees by applying force to the tab of one of the gears with a pair of forceps while the device was still inside of the well. This process was repeated three times until four arc lengths of the tube were seeded with fibroblast suspension. The assembly was then submerged in 1.2 mL DMEM/10% FBS/1% P/S by pipetting fresh media into the bottom of the well until the assembly was completely covered by media. After fibroblast cell attachment overnight, the assembly was removed intact from the well with a pair of forceps and placed upright, with the hollow gear resting on a capped hollow tube in an adjacent well of the 24-well plate. The solid gear was then removed from the assembly with a pair of forceps and discarded. Using the forceps, pressure was applied directly from above to the rod which forced the rod through hollow gear and into the capped hollow tube, resulting in the interior of the electrospun biopolymer tube being exposed. The entire device was then placed carefully into a polystyrene culture tube and a hydrogel-Schwann cell mixture was pipetted into the interior lumen of the electrospun biopolymer tube. Immediately after dispensing the cell mixture, 1.5 mL of culture media was added to the polystyrene tube to submerge the entire device. The tube was then capped and incubated at 37 °C and 5% CO2 for 20 minutes. After 20 minutes 1.0 mL of the media was replaced, and this process was repeated after 40 minutes. The culture media in the tube was replaced similarly every 2-3 days over the course of three weeks.

### Example 2

### Growing Schwann Cells on Fibers Using the Rectangular Microdevice

Rat Schwann cells were grown on E1001k fibers of approximately 50 μm using the rectangular device from FIG. 10. FIG. 12 is a 10× magnification imaging of the rat Schwann cells growing on the E1001k fibers.

While there have been described what are presently believed to be various aspects and certain desirable embodiments of the disclosure, those skilled in the art will recognize that changes and modifications may be made thereto without departing from the spirit of the disclosure, and it is intended to include all such changes and modifications as fall within the true scope of the disclosure.

Although the invention has been described in detail with reference to certain preferred embodiments, variations and modifications exist within the scope and spirit of the invention as described and defined in the following claims.

1. A snap-fit fixture comprising a large snap-fit fixture and a small snap-fit fixture, wherein the large snap-fit fixture and the small snap-fit fixture detachably and reversibly connect to create a platform.

2. The snap-fit fixture of claim 1, wherein the platform is configured to hold a biocompatible polymer material that can be seeded on one or both sides with cells.

3. The snap-fit fixture of claim 1, wherein the large snap-fit fixture and the small snap-fit fixture are made from polylactic acid (PLA), polycaprolactone (PCL), polyvinyl alcohol (PVA), or any combination thereof.

4. The snap-fit fixture of claim 1, wherein the large snap-fit fixture is about 2 mm to about 20 mm in height, and about 5 mm to about 40 mm in diameter.

5. The snap-fit fixture of claim 1, wherein the large snap-fit fixture comprises an aperture in the center of the large snap-fit fixture.

6. The snap-fit fixture of claim 1, wherein the small snap-fit fixture is about 2 mm to about 20 mm in height, and about 5 mm to about 40 mm in diameter.

7. The snap-fit fixture of claim 1, wherein the small snap-fit fixture comprises an aperture in the center of the small snap-fit fixture.

8. The snap-fit fixture of claim 1 having a weight of about 0.25 grams to about 5 grams.

9. The snap-fit fixture of claim 1, wherein the small snap-fit fixture comprises outwardly protruding tabs.

10. The snap-fit fixture of claim 1, wherein the large snap-fit fixture comprises outwardly protruding tabs.

11. The snap-fit fixture of claim 9, wherein the outwardly protruding tabs comprise insets.

12. The snap-fit fixture of claim 1, wherein the large snap-fit fixture comprises holes and/or openings that extend from the outside of the fixture into the interior space.

13. (canceled)

14. (canceled)

15. (canceled)

16. A 3D column microdevice comprising: a barrel, a center column, a gear-shaped ring with a central depression inset, and a gear-shaped ring with a patent opening.

17. The 3D column microdevice of claim 16, wherein the center column is capable of being inserted through a central hole in the gear-shaped ring; the ring is configured to slide down to the flange on the column; and a tubular biomaterial scaffold can be slid onto the column such that the column provides internal stability to a thin fiber of the scaffold material.

18. The 3D column microdevice of claim 16, wherein the gear-shaped ring with inset is configured to be snapped onto the uncapped end of the column.

19. The 3D column microdevice of claim 16 for seeding an exterior of a biodegradable polymer conduit with cells and for filling an interior luminal space of the conduit with macromolecules, growth factors, adhesion molecules, and/or cells.

20. (canceled)

21. A rectangular microdevice for seeding cells onto fibers comprising a rectangular body having inset recesses on one side of the body and a rectangular opening on the other side of the body.

22. The rectangular microdevice of claim 21, wherein the body is made from polylactic acid (PLA), polycaprolactone (PCL), polyvinyl alcohol (PVA), or any combination thereof.

23. The rectangular microdevice of claim 21, wherein the fibers are selected from the group comprising resorbable fibers, poly(lactic-co-glycolic acid) (PLGA) derived fibers, polyglycolic acid (PGA) derived fibers, a tyrosine-derived polymer with a high concentration of poly(ethylene glycol) (PEG), and any combination thereof.

24. The rectangular microdevice of claim 21, wherein the microdevice has a weight of about 1 gram to about 10 grams.

25. (canceled)
